(19) United States
(12) Patent Application Publication
(10) Pub. No.: US 2020/0173984 A1
(43) Pub. Date: Jun. 4, 2020

(54) CONVEYING PIPELINE, MANUFACTURING METHOD THEREOF, AND BIONIC PLATFORM

(71) Applicant: National Tsing Hua University, Hsinchu City (TW)

(72) Inventors: Fan-Gang Tseng, Hsinchu City (TW); Jye-Sheng Chen, Hsinchu City (TW); Jyun-Wei Chen, Hsinchu City (TW); Bo-Heng Liu, Hsinchu City (TW)

(73) Assignee: National Tsing Hua University, Hsinchu City (TW)

(21) Appl. No.: 16/417,635

(22) Filed: May 20, 2019

(30) Foreign Application Priority Data
Dec. 4, 2018 (TW) 107143413

(51) Int. Cl. G01N 33/50; B29C 63/42

(52) U.S. Cl. CPC G01N 33/5008; B29L 2023/22; B29C 63/42

(57) ABSTRACT

A conveying pipeline including a bionic tube, a delivery tube, and a shrinkable tube is provided. A portion of the delivery tube is located in the bionic tube, so that the bionic tube has an overlapping region with the delivery tube. The shrinkable tube wraps the bionic tube located in the overlapping region.

### CROSS-REFERENCE TO RELATED APPLICATION

This application claims the priority benefit of Taiwan application serial no. 107143413, filed on Dec. 4, 2018. The entirety of the above-mentioned patent application is hereby incorporated by reference herein and made a part of this specification.

### BACKGROUND OF THE INVENTION

### Field of the Invention

The invention relates to a pipeline and a manufacturing method thereof, and particularly relates to a conveying pipeline and a manufacturing method thereof.

### Description of Related Art

The process of new drug research and development includes drug exploration, preclinical animal trials and clinical trials for product development, and the new drug can be examined, registered and listed after the new drug has clinical efficacy. The overall development time of new drugs will take 10-15 years, and each stage will cost a lot of research funds. However, if the new drug is successfully developed and listed, the output value is also very large.

In the preclinical trial phase, the efficacy and safety of drug are demonstrated primarily on animals, and many animals are sacrificed at this stage for testing. Therefore, if there is a platform for human bionic tissue, it can be directly tested on the platform to reduce the sacrifice of animals and observe the effects on human cells.

### SUMMARY OF THE INVENTION

The invention provides a conveying pipeline and a manufacturing method thereof, which can produce a bionic platform simply and quickly.

The invention provides a bionic platform, which can effectively simulate the tissue in the human body to make the experimental test result more accurate.

The invention provides a conveying pipeline, which includes a bionic tube, a delivery tube, and a shrinkable tube. A portion of the delivery tube is located in the bionic tube, so that the bionic tube has an overlapping region with the delivery tube. The shrinkable tube wraps the bionic tube located in the overlapping region.

According to an embodiment of the invention, in the conveying pipeline, the material of the bionic tube is, for example, hydrogel, collagen, gelatin, hyaluronic acid, chitosan, chitin, alginic acid, cellulose or a derivative thereof.

According to an embodiment of the invention, in the conveying pipeline, the material of the delivery tube is, for example, a rigid material.

According to an embodiment of the invention, in the conveying pipeline, the delivery tube is, for example, a needle or a plastic pipe.

According to an embodiment of the invention, in the conveying pipeline, the shrinkable tube may be a heat-shrinkable tube or a cold-shrinkable tube.

According to an embodiment of the invention, in the conveying pipeline, the shrinkable tube may not wrap the bionic tube located outside the overlapping region.

According to an embodiment of the invention, in the conveying pipeline, the shrinkable tube may further wrap at least a portion of the delivery tube located outside the overlapping region.

According to an embodiment of the invention, the conveying pipeline may further include a filler. The filler may be located between the shrinkable tube and the bionic tube.

According to an embodiment of the invention, in the conveying pipeline, the filler may extend outside the shrinkable tube.

According to an embodiment of the invention, in the conveying pipeline, the filler may wrap at least a portion of an outer surface of the shrinkable tube.

According to an embodiment of the invention, in the conveying pipeline, the material of the filler is, for example, hydrogel, collagen, gelatin, hyaluronic acid, chitosan, chitin, alginic acid, cellulose or a derivative thereof.

The invention provides a bionic platform including the conveying pipeline.

The invention provides a method of manufacturing a conveying pipeline, which includes the following steps. A bionic tube is provided. A portion of a delivery tube is inserted into the bionic tube, so that the bionic tube has an overlapping region with the delivery tube. The bionic tube located in the overlapping region is wrapped with a shrinkable tube to fix the bionic tube to the delivery tube.

According to an embodiment of the invention, in the manufacturing method of the conveying pipeline, the shrinkable tube may not wrap the bionic tube located outside the overlapping region.

According to an embodiment of the invention, in the manufacturing method of the conveying pipeline, the shrinkable tube may wrap at least a portion of the delivery tube located outside the overlapping region.

According to an embodiment of the invention, in the manufacturing method of the conveying pipeline, when the shrinkable tube is a heat-shrinkable tube, the method of wrapping the bionic tube located in the overlapping region with the shrinkable tube may include the following Steps. The shrinkable tube is sleeved on the bionic tube located in the overlapping region. The shrinkable tube is heated to shrink, so that the shrinkable tube wraps the bionic tube located in the overlapping region.

According to an embodiment of the invention, in the manufacturing method of the conveying pipeline, when the shrinkable tube is a cold-shrinkable tube, the method of wrapping the bionic tube located in the overlapping region with the shrinkable tube may include the following Steps. The shrinkable tube is expanded, and the shrinkable tube is sleeved on the bionic tube located in the overlapping region. The shrinkable tube shrinks by elastic retractive force, so that the shrinkable tube wraps the bionic tube located in the overlapping region.

According to an embodiment of the invention, the manufacturing method of the conveying pipeline may further include filling a filler between the shrinkable tube and the bionic tube.

According to an embodiment of the invention, in the manufacturing method of the conveying pipeline, the filler may extend outside the shrinkable tube.

According to an embodiment of the invention, in the manufacturing method of the conveying pipeline, the filler may wrap at least a portion of an outer surface of the shrinkable tube.

Based on the above, in the conveying pipeline and the manufacturing method thereof according to the invention, since the bionic tube is fixed on the delivery tube by the connecting technology of the shrinkable tube, the bionic platform can be easily and quickly produced. Furthermore, in the bionic platform according to the invention, since the conveying pipeline has the bionic tube, the bionic platform can effectively simulate the tissue in the human body to make the experimental test result more accurate.

In order to make the aforementioned and other objects, features and advantages of the invention comprehensible, a preferred embodiment accompanied with figures is described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a schematic view of a conveying pipeline according to an embodiment of the invention.

FIG. 2 is a flow chart illustrating a manufacturing process of a conveying pipeline according to an embodiment of the invention.

FIG. 3 is a schematic view of a conveying pipeline according to another embodiment of the invention.

FIG. 4 is a schematic view of a conveying pipeline according to another embodiment of the invention.

FIG. 5 is a schematic view of a bionic platform according to an embodiment of the invention.

FIG. 6 is a schematic view of a bionic platform according to another embodiment of the invention.

### DESCRIPTION OF THE EMBODIMENTS

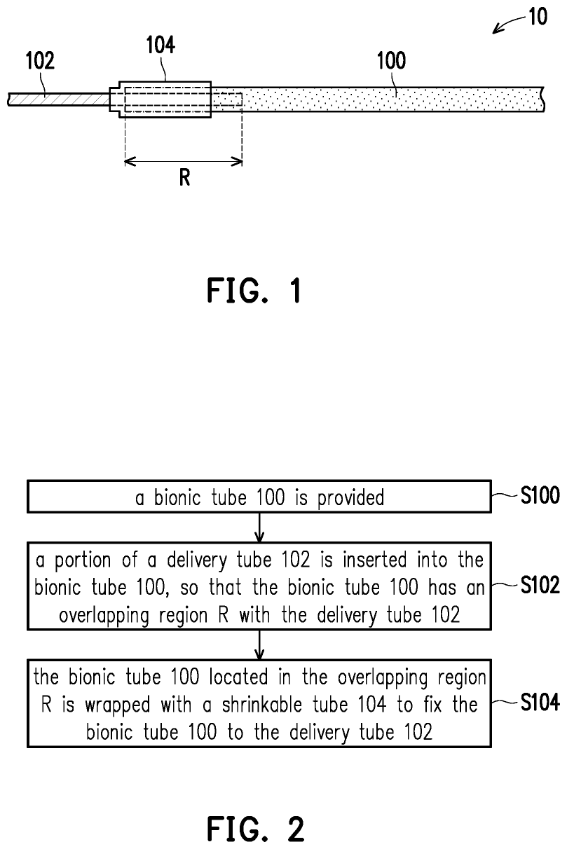

FIG. 1 is a schematic view of a conveying pipeline according to an embodiment of the invention. FIG. 2 is a flow chart illustrating a manufacturing process of a conveying pipeline according to an embodiment of the invention.

Referring to FIG. 1 and FIG. 2, step S100 is performed, a bionic tube 100 is provided. The bionic tube 100 may be a hollow tube. The material of the bionic tube 100 may be a highly biocompatible material, such as hydrogel, collagen, gelatin, hyaluronic acid, chitosan, chitin, alginic acid, cellulose or a derivative thereof.

Step S102 is performed, a portion of a delivery tube 102 is inserted into the bionic tube 100, so that the bionic tube 100 has an overlapping region R with the delivery tube 102. The material of the delivery tube 102 is, for example, a rigid material, such as stainless steel or plastic. The delivery tube 102 is, for example, a needle or a plastic tube.

Step S104 is performed, the bionic tube 100 located in the overlapping region R is wrapped with a shrinkable tube 104 to fix the bionic tube 100 to the delivery tube 102. Since the shrinkable tube 104 wraps the bionic tube 100 and applies pressure to the bionic tube 100 when the shrinkable tube 104 shrank, the bionic tube 100 can be fixed to the delivery tube 102 and leakage of fluid in the pipeline can be prevented. In addition, the bionic tube 100 is fixed on the delivery tube 102 by the connecting technology of the shrinkable tube 104, whereby the bionic platform can be easily and quickly produced.

Furthermore, the shrinkable tube 104 may not wrap the bionic tube 100 located outside the overlapping region R, whereby the shrinkable tube 104 can be prevented from applying pressure to the bionic tube 100 located outside the overlapping region R, so that the bionic tube 100 located outside the overlapping region R can be prevented from being deformed by pressure, but the invention is not limited thereto. Moreover, the shrinkable tube 104 may further wrap at least a portion of the delivery tube 102 located outside the overlapping region R, thereby further preventing leakage of fluid in the pipeline, but the invention is not limited thereto.

The shrinkable tube 104 may be a heat-shrinkable tube or a cold-shrinkable tube. The material of the heat-shrinkable tube is, for example, polyethylene (PE) or polyethylene terephthalate (PET). The material of the cold-shrinkable tube is, for example, a synthetic rubber, such as ethylene propylene rubber or silicon rubber. When the shrinkable tube 104 is the heat-shrinkable tube, the method of wrapping the bionic tube 100 located in the overlapping region R with the shrinkable tube 104 may include the following steps. The shrinkable tube 104 is sleeved on the bionic tube 100 located in the overlapping region R. The shrinkable tube 104 is heated to shrink, so that the shrinkable tube 104 wraps the bionic tube 100 located in the overlapping region R.

Moreover, when the shrinkable tube 104 is the cold-shrinkable tube, the method of wrapping the bionic tube 100 located in the overlapping region R with the shrinkable tube 104 may include the following steps. The shrinkable tube 104 is expanded, and the shrinkable tube 104 is sleeved on the bionic tube 100 located in the overlapping region R. The shrinkable tube 104 shrinks by an elastic retractive force, so that the shrinkable tube 104 wraps the bionic tube 100 located in the overlapping region R.

Hereinafter, the conveying pipeline 10 of the above embodiment will be described with reference to FIG. 1. In addition, although the method of forming the conveying pipeline 10 is described by taking the above method as an example, the invention is not limited thereto.

Referring to FIG. 1, the conveying pipeline 10 includes a bionic tube 100, a delivery tube 102, and a shrinkable tube 104. A portion of the delivery tube 102 is located in the bionic tube 100, so that the bionic tube 100 has an overlapping region R with the delivery tube 102. The shrinkable tube 104 wraps the bionic tube 100 located in the overlapping region R. The shrinkable slide tube 104 may not wrap the bionic tube 100 located outside the overlapping region R. The shrinkable tube 104 may further wrap at least a portion of the delivery tube 102 located outside the overlapping region R. In addition, the material, the arrangement, and the effect of each component in conveying the pipeline 10 have been described in detail in the above embodiments, and the description thereof is not repeated herein.

Based on the above embodiment, in the conveying pipeline 10 and the manufacturing method thereof, since the bionic tube 100 is fixed on the delivery tube 102 by the connecting technology of the shrinkable tube 104, the bionic platform can be easily and quickly produced. For example, cells can be injected into the bionic tube 100, thereby producing a hollow cell tube used to be a platform for various tests. Therefore, the conveying pipeline 10 can transform the cells originally cultured in two dimensions into a three-dimensional bionic tube structure, so that the characteristics of the cell culture environment can be more similar to the organs in the real human body, and different drugs can be injected into the hollow cell tube for testing. In this way, the use of animal experiments can be reduced, and the response of human cells to drugs can be further understood.

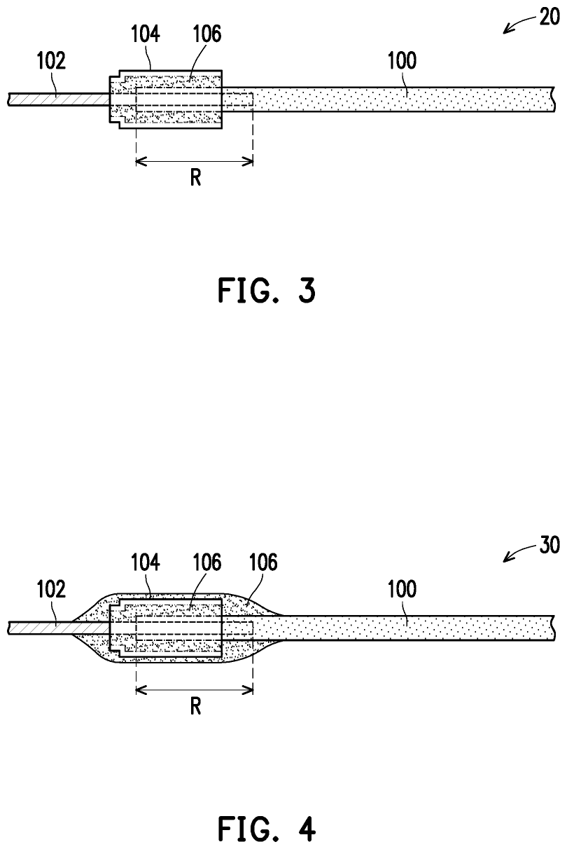

FIG. 3 is a schematic view of a conveying pipeline according to another embodiment of the invention.

Referring to FIG. 1 and FIG. 3, the difference between the conveying pipeline 20 of FIG. 3 and the conveying pipeline 10 of FIG. 1 is described as follows. The conveying pipeline 20 further includes a filler 106. The filler 106 may be located between the shrinkable tube 104 and the bionic tube 100. By filling the filler 106 between the shrinkable tube 104 and the bionic tube 100, the pressure applied by the shrinkable tube 104 on the bionic tube 100 located in the overlapping region R can be increased, thereby improving the leak-proof capability of the pipeline. In addition, the filler 106 may be located between the shrinkable tube 104 and the delivery tube 102 located outside the overlapping region R, thereby further improving the leak-proof capability of the pipeline. The material of the filler 106 is, for example, hydrogel, collagen, gelatin, hyaluronic acid, chitosan, chitin, alginic acid, cellulose or a derivative thereof.

Furthermore, the difference between the manufacturing methods of the conveying pipeline 20 and the conveying pipeline 10 is described as follows. The method of manufacturing the conveying pipeline 20 further includes filling the filler 106 between the shrinkable tube 104 and the bionic tube 100. In one embodiment, the filler 106 may wrap the bionic tube 100 located in the overlapping region R, and then the shrinkable tube 104 is sleeved on the bionic tube 100 located in the overlapping region R, so that the filler 106 is filled between the shrinkable tube 104 and the bionic tube 100. Next, the shrinkable tube 104 shrinks to wrap the bionic tube 100 located in the overlapping region R. In another embodiment, the shrinkable tube 104 may be sleeved on the bionic tube 100 located in the overlapping region R, and then the filler 106 may be filled between the shrinkable tube 104 and the bionic tube 100. Next, the shrinkable tube 104 shrinks to wrap the bionic tube 100 located in the overlapping region R. In addition, the manufacturing method of the conveying pipeline 20 may further include filling the filler 106 between the shrinkable tube 104 and the delivery tube 102 located outside the overlapping region R.

Moreover, the same components in the conveying pipeline 20 and the conveying pipeline 10 are denoted by the same reference numerals and the description thereof is omitted.

Based on the above embodiment, in the conveying pipeline 20, the filler 106 can be located between the shrinkable tube 104 and the bionic tube 100, whereby the pressure applied by the shrinkable tube 104 on the bionic tube 100 located in the overlapping region R can be increased. Therefore, the leak-proof capability of the pipeline can be improved.

FIG. 4 is a schematic view of a conveying pipeline according to another embodiment of the invention.

Referring to FIG. 3 and FIG. 4, the difference between the conveying pipeline 30 of FIG. 4 and the conveying pipeline 20 of FIG. 3 is described as follows. In the conveying pipeline 30, the filler 106 may extend outside the shrinkable tube 104, thereby further enhancing the leak-proof capability of the pipeline. For example, the filler 106 may extend outside of the shrinkable tube 104 to wrap at least one of the bionic tube 100 and the delivery tube 102 both located outside of the shrinkable tube 104. Additionally, the filler 106 may wrap at least a portion of the outer surface of the shrinkable tube 104, thereby further enhancing the leak-proof capability of the pipeline. Moreover, the same components in the conveying pipeline 30 and the conveying pipeline 20 are denoted by the same reference numerals and the description thereof is omitted.

On the other hand, the conveying pipelines in the above embodiments can be applied to various bionic platforms. That is, the bionic platform may include the above-described conveying pipeline, as exemplified below.

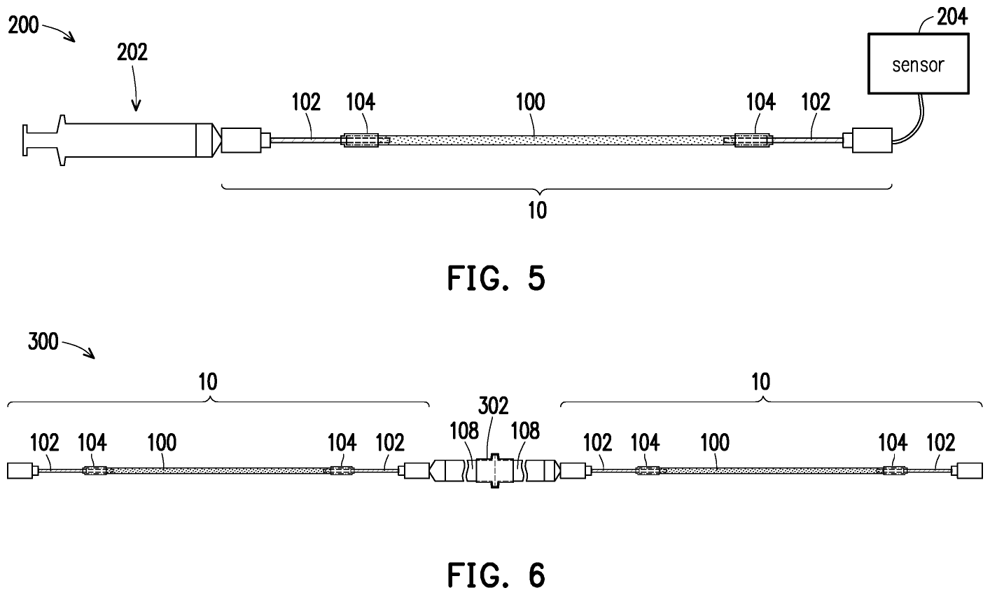

FIG. 5 is a schematic view of a bionic platform according to an embodiment of the invention.

Referring to FIG. 5, the bionic platform 200 may include a conveying pipeline 10, a syringe 202, and a sensor 204. A conveying pipeline 10 is connected between the syringe 202 and the sensor 204. In the present embodiment, the delivery tube 102 in the conveying pipeline 10 is exemplified by a needle. The delivery tube 102 on one side of the conveying pipeline 10 may be a needle of the syringe 202. The syringe 202 can be used to inject a variety of different medicaments into the conveying pipeline 10. The sensor 204 is, for example, a pressure gauge, but the invention is not limited thereto. The type of sensor 204 can be determined by the parameters actually to be measured. In addition, other components in the conveying pipeline 10 can be referred to the description of the above embodiment, and the description thereof is not repeated herein.

For example, when the bionic platform 200 is a platform for simulating a glomerular structure, the podocytes may be injected into the bionic tube 100 to produce a hollow cell tube. Next, a variety of different medicaments may be injected into the conveying pipeline 10 by the syringe 202. Furthermore, the syringe 202 may provide pressure to the bionic tube 100 to simulate the glomerular environment, and the pressure value may be measured by the sensor 204. In this way, the bionic platform 200 can have a glomerular environment more similar to the real human body, thereby better understanding the response of human cells to drugs.

Based on the above embodiment, in the bionic platform 200, since the conveying pipeline 10 has the bionic tube 100, the bionic platform 200 can effectively simulate the tissue in the human body to make the experimental test result more accurate.

FIG. 6 is a schematic view of a bionic platform according to another embodiment of the invention.

Referring to FIG. 6, the bionic platform 300 may include a conveying pipeline 10 and a shrinkable tube 302. The conveying pipeline 10 may further include a needle cylinder 108. In the present embodiment, the delivery tube 102 in the conveying pipeline 10 is exemplified by a needle. The delivery tube 102 of the conveying pipeline 10 may be a needle connected to the needle cylinder 108. The shrinkable tube 302 can connect two adjacent needle cylinders 108 to communicate adjacent two conveying pipelines 10. The shrinkable tube 302 may be a heat-shrinkable tube or a cold-shrinkable tube. In one embodiment, the shrinkable tube 302 may be sleeved on the adjacent two needle cylinders 108, and then the shrinkable tube 302 may shrink to connect the adjacent two needle cylinders 108. In addition, other components in the conveying pipeline 10 can be referred to the description of the above embodiment, and the description thereof is not repeated here.

For example, when the bionic platform 300 is a platform for simulating a heart environment, the myocardial cells may be injected into the bionic tube 100 to produce a single-layer tubular myocardial tissue. It can be seen from the experimental results that the growth of the single-layer tubular myocardial tissue cultured in the bionic tube 100 conforms to the geometrical specification, and the myocardial cells have synchronized self-pulsation. Then, the conveying pipelines 10 may be communicated by the shrinkable tube 302, whereby the bionic platform 300 can be formed. Next, a variety of different medicaments may be injected into the conveying pipeline 10. In this way, the bionic platform 300 can have a heart environment more similar to the real human body, thereby better understanding the response of human cells to drugs. In addition, the delivery tube 102 can be further connected to a pump (not shown) to push the liquid in the conveying pipeline 10, but the invention is not limited thereto. Those skilled in the art can determine whether the bionic platform 300 needs to use a pump according to the experimental design requirements.

Based on the above embodiment, in the bionic platform 300, since the conveying pipeline 10 has the bionic tube 100, the bionic platform 300 can effectively simulate the tissue in the human body to make the experimental test result more accurate.

In the above embodiment, although the conveying pipeline 10 is exemplified by being applied to the bionic platform 200 and the bionic platform 300, the invention is not limited thereto. As long as the bionic platform includes the conveying pipeline 10, it falls within the scope covered by the invention.

Furthermore, although the conveying pipeline in the bionic platform 200 and the bionic platform 300 is exemplified by the conveying pipeline 10, the invention is not limited thereto. In other embodiments, the conveying pipeline 10 may be replaced by the conveying pipeline 20 or the conveying pipeline 30.

In summary, in the conveying pipeline of the aforementioned embodiment and the manufacturing method thereof, since the bionic tube is fixed on the delivery tube by the connecting technology of the shrinkable tube, the bionic platform can be easily and quickly produced. Furthermore, in the bionic platform of the aforementioned embodiment, since the conveying pipeline has the bionic tube, the bionic platform can effectively simulate the tissue in the human body to make the experimental test result more accurate.

Although the invention has been described with reference to the above embodiments, it will be apparent to one of ordinary skill in the art that modifications to the described embodiments may be made without departing from the spirit of the invention. Accordingly, the scope of the invention is defined by the attached claims not by the above detailed descriptions.

What is claimed is:

1. A conveying pipeline comprising:
a bionic tube;
a delivery tube, wherein a portion of the delivery tube is located in the bionic tube, so that the bionic tube has an overlapping region with the delivery tube; and
a shrinkable tube wrapping the bionic tube located in the overlapping region.

2. The conveying pipeline according to claim 1, wherein a material of the bionic tube comprises hydrogel, collagen, gelatin, hyaluronic acid, chitosan, chitin, alginic acid, cellulose or a derivative thereof.

3. The conveying pipeline according to claim 1, wherein a material of the delivery tube comprises a rigid material.

4. The conveying pipeline according to claim 3, wherein the delivery tube comprises a needle or a plastic tube.

5. The conveying pipeline according to claim 1, wherein the shrinkable tube comprises a heat-shrinkable tube or a cold-shrinkable tube.

6. The conveying pipeline according to claim 1, wherein the shrinkable tube does not wrap the bionic tube located outside the overlapping region.

7. The conveying pipeline according to claim 1, wherein the shrinkable tube further wraps at least a portion of the delivery tube located outside the overlapping region.

8. The conveying pipeline according to claim 1, further comprising a filler, wherein the filler is located between the shrinkable tube and the bionic tube.

9. The conveying pipeline according to claim 8, wherein the filler extends outside the shrinkable tube.

10. The conveying pipeline according to claim 9, wherein the filler wraps at least a portion of an outer surface of the shrinkable tube.

11. The conveying pipeline according to claim 8, wherein a material of the filler comprises hydrogel, collagen, gelatin, hyaluronic acid, chitosan, chitin, alginic acid, cellulose or a derivative thereof.

12. A bionic platform comprising the conveying pipeline according to claim 1.

13. A method of manufacturing a conveying pipeline, comprising:
providing a bionic tube;
inserting a portion of a delivery tube into the bionic tube, so that the bionic tube has an overlapping region with the delivery tube; and
wrapping the bionic tube located in the overlapping region with a shrinkable tube to fix the bionic tube to the delivery tube.

14. The method of manufacturing the conveying pipeline according to claim 13, wherein the shrinkable tube does not wrap the bionic tube located outside the overlapping region.

15. The method of manufacturing the conveying pipeline according to claim 13, wherein the shrinkable tube further wraps at least a portion of the delivery tube located outside the overlapping region.

16. The method of manufacturing the conveying pipeline according to claim 13, wherein when the shrinkable tube is a heat-shrinkable tube, a method of wrapping the bionic tube located in the overlapping region with the shrinkable tube comprises:
sleeving the shrinkable tube on the bionic tube located in the overlapping region; and
heating the shrinkable tube to shrink, so that the shrinkable tube wraps the bionic tube located in the overlapping region.

17. The method of manufacturing the conveying pipeline according to claim 13, wherein when the shrinkable tube is a cold-shrinkable tube, a method of wrapping the bionic tube located in the overlapping region with the shrinkable tube comprises:
expanding the shrinkable tube and sleeving the shrinkable tube on the bionic tube located in the overlapping region; and
shrinking the shrinkable tube by elastic retractive force, so that the shrinkable tube wraps the bionic tube located in the overlapping region.

18. The method of manufacturing the conveying pipeline according to claim 13, further comprising filling a filler between the shrinkable tube and the bionic tube.

19. The method of manufacturing the conveying pipeline according to claim 18, wherein the filler extends outside the shrinkable tube.

20. The method of manufacturing the conveying pipeline according to claim 19, wherein the filler wraps at least a portion of an outer surface of the shrinkable tube.
